(19) United States
(12) Patent Application Publication
(10) Pub. No.: US 2026/0198791 A1
(43) Pub. Date: Jul. 16, 2026

(54) MACHINE LEARNING MODEL TO ESTIMATE SYSTOLIC AND DIASTOLIC BLOOD PRESSURE FROM ACQUIRED BLOOD PRESSURE DATA

(71) Applicant: 42 Health Sensor Holdings LTD, Calgary (CA)

(72) Inventors: Fatima Chakir, Calgary (CA); Robert Stanley Sheldon, Calgary (CA); Daniel Edward Roach, Calgary (CA)

(21) Appl. No.: 19/449,775

(22) Filed: Jan. 15, 2026

Related U.S. Application Data
(60) Provisional application No. 63/745,399, filed on Jan. 15, 2025.

(51) Int. Cl. A61B 5/022; A61B 5/021; G16H 50/30

(52) U.S. Cl. CPC A61B 5/02225; A61B 5/02108; A61B 5/02141; G16H 50/30

(57) ABSTRACT

A wearable ear-mounted cardiovascular device acquires blood pressure data while applying controlled confining pressure to ear tissue and sensing arterial pulsatility with a force sensor. The waveform data are processed to extract multiple features from inflation, deflation, compression, and decompression segments, including pressures at characteristic changes in oscillation trend, oscillation amplitudes, frequency-domain descriptors, and waveform-derived statistics. Separate machine learning regression models, such as Support Vector Machines, use distinct feature sets to estimate systolic and diastolic blood pressure relative to reference arm cuff measurements. The trained models provide real-time, cuffless blood pressure values from the ear device, and can operate with stepwise compression or in a passive ear-clip configuration calibrated intermittently using external blood pressure devices.

### CROSS-REFERENCE TO RELATED APPLICATION

This application claims priority to U.S. Provisional Patent Application No. 63/745,399, filed Jan. 15, 2025, entitled “MACHINE LEARNING MODEL TO ESTIMATE SYSTOLIC AND DIASTOLIC BLOOD PRESSURE FROM BLOOD PRESSURE WAVEFORMS USING A NOVEL CUFFLESS DEVICE,” the disclosure of which is expressly incorporated herein by reference in its entirety.

### BACKGROUND

In order to diagnose or monitor a patient experiencing syncope or hypertension, a physician requires patient blood pressure (BP) data. A measurement taken by the physician during an in-clinic appointment captures only the patient's blood pressure at the time of the reading and does not detect transient changes in blood pressure throughout a typical day. Often the one-time reading is insufficient, and thus BP data collected over an extended period is ordered by the physician. Typically, this data is collected using an ambulatory blood pressure (BP) monitor over a period of 24 hours.

A standard ambulatory BP monitor consists of an automatically inflatable cuff worn on the user's arm for the diagnostic period, connected by an air supply tube to a monitoring device. These monitors are cumbersome to wear and interfere with normal activity as they automatically inflate and squeeze the user's arm to measure blood pressure approximately every 15 to 30 minutes of the day. It is recommended that the user limit movement and sit down, if possible, when the cuff is inflating and taking a reading. Disadvantageously, a user may not experience a typical day when interrupted regularly with an inflating cuff. Additionally, with measurements taken at a specified time interval, the data collected is a series of individual measurements, rather than a continuous, uninterrupted stream of data, thereby not truly providing full blood pressure data over the monitoring period for the patient.

### SUMMARY

A wearable ear-mounted cardiovascular device acquires blood pressure data while applying controlled confining pressure to ear tissue and sensing arterial pulsatility with a force sensor. The waveform data are processed to extract multiple features from inflation, deflation, compression, and/or decompression segments, including pressures at characteristic changes in oscillation trend, oscillation amplitudes, frequency-domain descriptors, and waveform-derived statistics. Separate machine learning regression models, such as Support Vector Machines, use distinct feature sets to estimate systolic and diastolic blood pressure relative to reference arm cuff measurements. The trained models provide real-time, cuffless blood pressure values from the ear device, and can operate with stepwise compression or in a passive ear-clip configuration calibrated intermittently using external blood pressure devices.

According to an aspect, a method for determining blood pressure is disclosed that includes receiving, by one or more processors, blood pressure data acquired from a wearable cardiovascular monitoring device configured to be worn on an ear of the subject; extracting, from the blood pressure data, a plurality of waveform-based features; determining a systolic blood pressure value of the subject by inputting at least a subset of the waveform-based features into a machine learning-based systolic blood pressure model; and determining a diastolic blood pressure value of the subject by inputting at least a subset of the waveform-based features into a machine learning-based diastolic blood pressure model.

In another aspect, a system for determining blood pressure is disclosed that includes a wearable cardiovascular monitoring device configured to be worn on an ear of the subject and to generate blood pressure data; and one or more processors coupled to a memory storing instructions which, when executed by the one or more processors, cause the system to receive the blood pressure data from the wearable cardiovascular monitoring device; extract a plurality of waveform-based features from the blood pressure data; determine a systolic blood pressure value using a machine learning-based systolic blood pressure model applied to at least a subset of the waveform-based features; and determine a diastolic blood pressure value using a machine learning-based diastolic blood pressure model applied to at least a subset of the waveform-based features.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of illustrative embodiments, is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIG. 1 is a diagram of an implementation of a system for blood pressure measurement with a wearable cardiovascular monitoring device;

FIG. 2 is a chart that shows example visco-elastic responses to instant changes in ear-vise strain;

FIG. 3 is a chart that shows an example of three cycles of ear-vise stop-and-go compression and decompression on an incompressible steel disk;

FIG. 4 is a chart that shows an example arterial blood pressure data superimposed on stress-relaxation kinetics created by visco-elastic response to instant strain increases created by ear-vise compression of ear tissue;

FIG. 5 is a chart that shows an example sketch of visco-elastic moto-phase sampling (VEMPS) methodology for sampling arterial blood pressure in phase with pulse width modulation (PWM);

FIG. 6 is a chart of an example signal processing methodology that could be used to precisely locate the fiduciary points;

FIGS. 7A, 7B and 7C illustrate determining systolic blood pressure (SBP) and diastolic blood pressure (DBP) by systematically performing a stepwise compression/decompression of ear tissue while measuring predetermined waveform parameters at each step;

FIG. 8 is an operational flow of another implementation of a method for blood pressure measurement with a wearable cardiovascular monitoring device; and

FIG. 9 illustrates an example of recorded inflation and deflation and/or compression and decompression during confining blood pressure;

FIG. 10 illustrates an example scatter plot of SBP model estimates;

FIG. 11 illustrates an example scatter plot of DBP model estimates;

FIG. 12 illustrates an example Bland Atman plot of SBP model estimates;

FIG. 13 illustrates an example Bland Atman plot of DBP model estimates; and

FIG. 14 shows an exemplary computing environment in which example embodiments and aspects may be implemented.

### DETAILED DESCRIPTION

Compared to the fingertip, wrist and upper arm, the ear is a very stable place to detect arterial blood pressure (BP). Ears do not swing around when a subject walks or exercises, ears do not experience signal artifact due to muscle activation/deactivation, and the arch of cartilaginous connection to the head provides an out-of-the-way stable attachment structure. Moreover, most ears manifest a planar cartilage element located somewhere in the vicinity of pulsating arteries. The planar cartilage element need not be large, only large enough to accommodate the planar compression of the arteries across the ear tissue. Curved cartilage presents a greater challenge, since any trans ear tissue compression in the presence of curved cartilage will result in differential compression as the planar face of the compression device attempts to uniformly compress the curved cartilage.

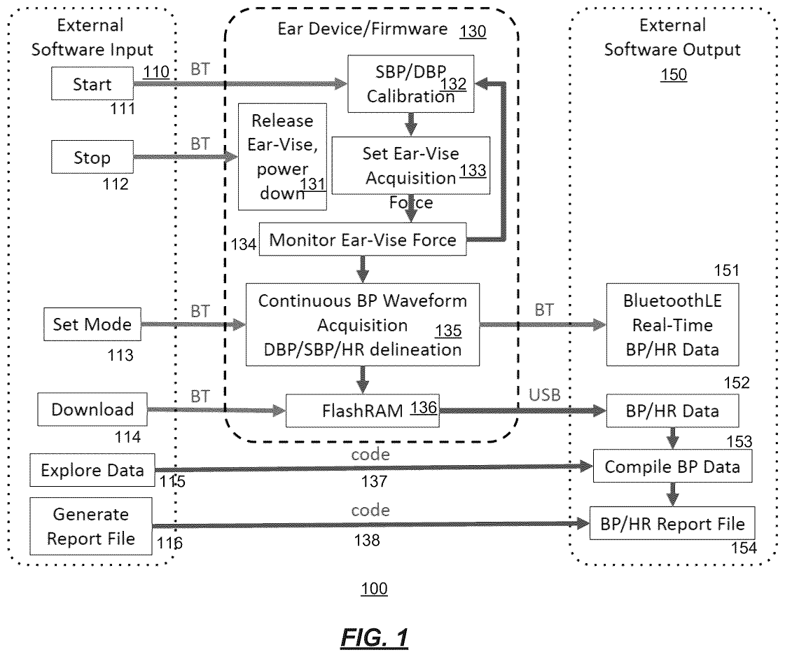

FIG. 1 is a diagram of an implementation of a system 100 for blood pressure measurement with a wearable cardiovascular monitoring device. The system comprises external software input 110, an ear device (firmware) 130, and external software output 150.

The external software input 110 may comprise inputs to the ear device 130 such as start 111, stop 112, set mode 113, download 114, explore data 115, and generate report file 116, for example, though these are not intended to be limiting. The inputs to the ear device 130 are not limited to those listed, and may comprise more or fewer inputs depending on the implementation. The inputs may be provided to the ear device (firmware) 130 using BluetoothLE (Bluetooth Low Energy, also referred to herein at BT).

The ear device 130 may comprise a release ear-vise, power down module 131, a systolic blood pressure (SBP)/diastolic blood pressure (DBP) calibration module 132, a set ear-vise acquisition module 133, a monitor ear-vise force module 134, a continuous blood pressure acquisition with DBP/SBP/heart rate (HR) delineation module 135, and flash memory such as flash RAM 136, for example, though these are not intended to be limiting. The ear device 103 may comprise more or fewer modules and/or components than those listed, depending on the implementation.

The SBP/DBP calibration module 132 may be actuated or started pursuant to receiving the start 111 input. The release ear-vise, power down module 131 may be actuated or started pursuant to receiving the stop 112 input.

After the SBP/DBP calibration module 132 performs calibration of the ear device 130, the set ear-vise acquisition module 133 is actuated or started which then actuates or starts the monitor ear-vise force module 134. The continuous BP acquisition with DBP/SBP/HR delineation module 135 is then begun, followed by storage of the acquired data in the flash RAM 136. The SBP/DBP calibration module may also provide inputs to the ear device 130 firmware to enable a stepwise compression/decompression functionality, as described below.

The external software output 150 may comprise BT real-time BP/HR data 151 received from the continuous BP acquisition with DBP/SBP/HR delineation module 135. The BluetoothLE real-time BP/HR data 151 may be obtained or otherwise received or retrieved pursuant to the set mode 113 input of the external software input 110, which may provide instructions to the ear device 130, and thus the a continuous BP acquisition with DBP/SBP/HR delineation module 135, by BT in some implementations.

The external software output 150 may also receive BP/HR data 152 from storage of the ear device 130, such as from the flash RAM 136 via USB. The BP/HR data 152 may be obtained or otherwise received or retrieved pursuant to the download 114 input of the external software input 110, which may provide instructions to the ear device 130, and thus the flash RAM 136, by BT in some implementations.

The BP data from the BP/HR data 152 may be compiled into compiled BP data 153 (e.g., pursuant to the explore data 115 input of the external software input 110 using code 137 to compile the BP data).

Returning to FIG. 1, A BP/HR report file 154 may be generated (e.g., pursuant to the generate report file 116 input of the external software input 110 using code 138 to generate the BP/HR report file 154) and stored and/or otherwise outputted.

A sensing technique monitors the pulsatility of some measured parameter as the confining pressure is varied. In most instances, the compression drive is programmed to cause a steady increase in tissue confining compression until the pulsatility of the measured parameter is statistically eliminated (i.e., BP occlusion), and then a little more. At this point, the confining pressure exceeds the arterial systolic BP by 20-50 mmHg; upon the slow controlled decrease in confining pressure, the systolic BP eventually exceeds the decreasing confining pressure and the pulsatility becomes detectable again. This point is used in determining the systolic BP. As the confining pressure further decreases, the magnitude of the pulsatility (or oscillations) increases eventually reaching a maximum peak to trough magnitude. Empirically, this unique point is used to establish the diastolic BP. As the confining pressure further decreases, the pulsatility magnitude slowly decreases until the confining pressure reaches zero. At this point, the diastolic and systolic pressures are determined. For continuous blood pressure measurements, some comfortable confining pressure is applied to the tissue, and a continuous pulsatile trace is obtained. Thus, immediately one can correlate the just determined systole and diastole to the diastolic point and systolic points on the continuous trace. For as long as the trace pulsatile signal stays well-behaved, and does not deviate too drastically in shape or magnitude, then it is possible to estimate the continuous BP. When large signal aberrations occur in the pulsatile signal (typically at times of posture changes, large skeletal muscle activation, and respiratory changes), these deviations are detected via signal processing and a new calibration is automatically started, or a request for a new calibration is generated to the user.

There are many potential pulsatile signals and techniques that respond in a manner for use in oscillometric BP determination. If the ear-vise is sufficiently flexible (e.g., allowing for approximately 100 micron pulsatile displacement under normal BP conditions), then this micron displacement can be measured in several ways. These displacement techniques all rely on the ability to convert micro displacement to force through some sort of known stress/strain relationship. Typically, some elements of the ear-vise assembly, such as one of the vise faces, is disposed or mounted such that the compressive force from the gearbox motor and from pulsatile BP changes cause micron scale displacements that are then converted to a voltage proportional to force. In some embodiments, the displacement is measured across the compressed ear tissue. In other embodiments, the measured displacements can be on one side of the ear (e.g., behind an ear-vise face).

In an implementation, a Hall Effect Transducer (HET) may be disposed on one side of the ear tissue and coaxially coupled to the magnet dipole of a disk magnet on the other side of the ear tissue. As the distance between the disk magnet and the HET changes via the gearbox motor and the changing BP, the HET produces a change in voltage proportional to the change in detected magnetic field strength. This is a straightforward technique to implement, and requires no sophisticated radio-frequency (RF) electronics for signal detection, thus it is relatively low power. The Hall Effect Transducer route is feasible, but it has a larger footprint, consumes more current when active (e.g., 2 mA), and requires a manufacturing step for calibration.

In an implementation, as the BP varies in a pulsatile manner, it is capable of expanding arteries and ear tissue beds. Collectively, within the arterial bed under consideration, the expansion and contraction of arteries can exert an average force change on the ear-vise faces. This changing force can be converted into a voltage signal that can be sampled in many ways, including a MEMS force gauge. A micro-electrical mechanical systems (MEMS) force gauge is a much less expensive and smaller version of the Wheatstone bridge force gauge. These are smaller and much less expensive, and they exhibit similar thermal stability. Because they are an order of magnitude smaller than a traditional strain gauge, these MEMS force gauges exhibit reduced self-deformation characteristics, and reduced switching/settling times.

Real-time, miniaturized hardware and signal processing are making tremendous strides with the advent of specialized processing modules. Thus, there will be a growing use of sophisticated signal processing. In some implementations, ear-mounted BP units will have no macro moving parts at all, and the signal is derived from tiny sensors and sophisticated real-time signal processing. Alternatively, other ear-mounted platforms offer a quick macro-mechanical solution for measuring continuous blood pressure. In some implementations, a micro-processor controlled motor/gearbox/worm gear linear-actuator assembly is used to control the mechanical aspect of the ear tissue under investigation. As listed above, there are many sensors which can be used to detect the BP pulsatility under varying confining pressures. Another consideration for ear-based BP measurement is power consumption. In some implementations, the gearbox motor is only used during calibration, and thus is not a long-term drain on the battery. It is also important for the sensor package not to consume too much power. The sensors with the least current drain are the MEMS force sensor and the larger sized Wheatstone bridge strain gauges. These are passive resistive sensors; they have rapid turn-on times and can thus be turned off during intervals between samples, and these devices draw micro-amps. These force sensors are immune to changing ambient light conditions that can severely affect optical sensors. These force sensors inherently adjust for changing temperatures. The MEMS force sensor also has the smallest footprint and is pre-calibrated.

Furthermore, the device may be combined with one or more other sensors for use in monitoring the status of a patient including breathing rate, oxygen saturation, activity level, temperature and whether the patient is awake or sleeping. Examples of such additional sensors may include without limitation, oximeters, tricolor LED/phototransistors, accelerometers, microphones, nasal thermistors, thermometers, skin galvanometers, linear photo transistor arrays, ultrasonic probes and arrays, infra-red LED/phototransistor pairs, RF impedance plethysmography and piezoelectric vibration elements.

Advantageously, when the apparatus as set out above is applied to the ear, it is known that the ear does not include any muscle therein. Additionally, the ear is not located at an extremity of the body which is significantly subjected to movement as is a hand or foot. Therefore, the measurements obtained from such location will not be dependent upon the movement and activity of the user.

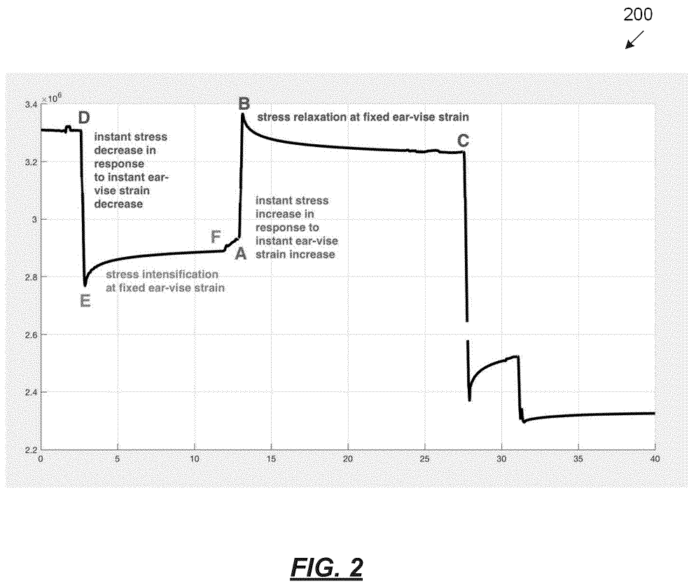

FIG. 2 is a chart 200 that shows example visco-elastic responses to instant changes in ear-vise strain using a device, for example, in some implementations of the ear device 130 of FIG. 1 that include a polymer visco-elastic clip. Although FIG. 2 is described with respect to some implementations of the device 130 of FIG. 1, this is not intended to be limiting, as the techniques described with respect to FIG. 2 may be used with any wearable cardiovascular monitoring device contemplated herein. More particularly, compression/decompression tests performed by the ear-vise on an incompressible steel disk displayed visco-elastic response behavior as seen in FIG. 2.

In FIG. 2, at point A, a DC motor of the ear device 130 is switched on briefly causing the ear-vise to rapidly increase the stress across the steel disk until such time that the motor is switched off at point B. At point B, the measured stress begins to immediately decay, while the motor is switched off. A high gear ratio of a planetary gearbox (136:1) and a worm gear (20:1) ensure that the ear-vise position remains locked when the power to the motor is removed. Thus, the observed stress decay kinetic is due to the visco-elastic response behavior of a polymer associated with the visco-elastic clip, which is the most readably deformable piece of polymer subject to the compression forces of the ear-vise; thus, FIG. 2 represents the visco-elastic response behavior of the visco-elastic clip. Note that the compression forces exponentially decay towards an asymptotic value at point C. This stress relaxation is a common response of polymers to rapidly applied strain.

At point D, the DC motor is switched on, in reverse, briefly causing the ear-vise to rapidly decrease the strain on the visco-elastic clip. At point E, the motor is switched off, the ear-vise position is locked, and there is a spontaneous intensification of the measured stress; it increases asymptotically towards point F. Note that the kinetic of stress intensification (although opposite in sign) closely matches the kinetic of the stress relaxation. In this instance, this intensification is attributed to polymer memory, where the polymer is slowly trying to revert to its former shape, thus increasing the stress. By numerically comparing the decay kinetics of stress relaxation vs. the kinetics of stress intensification, it was found that both kinetics were best fit by a bi-exponential decay process. Moreover, the decay coefficients of both were the same.

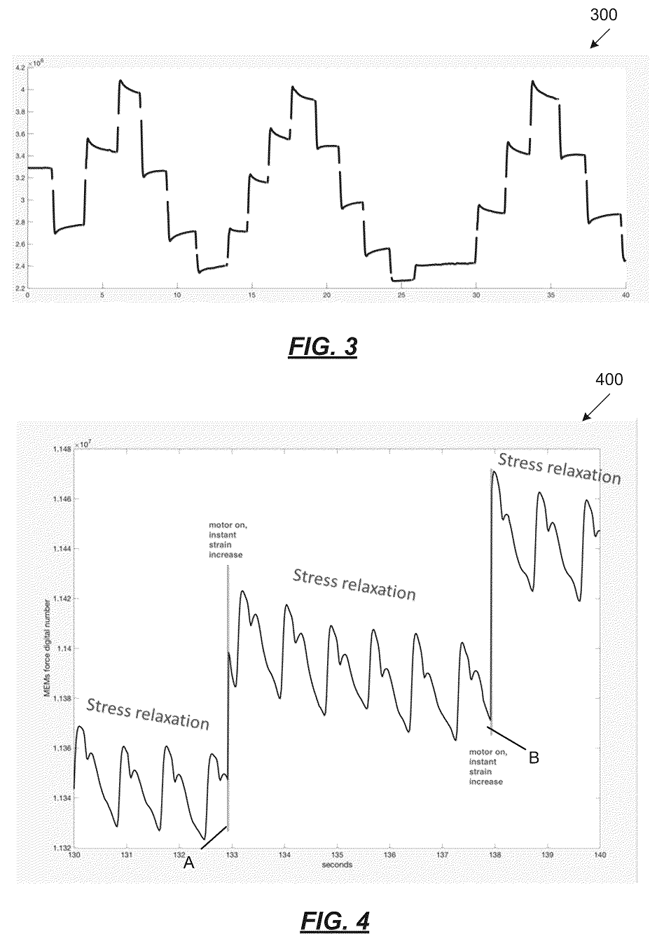

FIG. 3 is a chart 300 that shows an example of three cycles of ear-vise stop-and-go compression and decompression on an incompressible steel disk. In other words, FIG. 3 shows three cycles of stop-and-go stress increase followed by stop-and-go stress decrease, as the ear-vise attempts to strain the incompressible steel disk. Note that the visco-elastic kinetics always manifest when the motor is stopped. Immediately after rapid compression, the spontaneous visco-elastic response is relaxation; after rapid decompression the spontaneous visco-elastic response is intensification.

FIG. 4 is a chart 400 that shows an example arterial blood pressure data superimposed on stress-relaxation kinetics created by visco-elastic response to instant strain increases created by ear-vise compression of ear tissue. More particularly, FIG. 4 shows the stresses as measured when the ear-vise is placed on the ear and the motor is run briefly and then stopped for several seconds. Points A and B show the locations where the motor was turned on briefly. Note that the arterial blood pressure data is clearly superimposed upon the background stress relaxation kinetic.

Most oscillometric blood pressure measuring devices utilize a pneumatic cuff as the clamping mechanism for increasing and decreasing the compression pressure on the pulsatile tissue. A small pneumatic pump inflates the cuff to increase compression pressure, and an electrically operated air release valve controls the rate of deflation. These devices typically inflate at one speed; this is followed by a slower deflation of the cuff during which time the precise pulsatility measurements are acquired for assessment of systolic and diastolic pressure. Cuff deflation via the air release valve is continuous in nature; this continuity presents a smoothly continuously decreasing background pressure signal, upon which is superimposed the pulsatile waveform of the arterial blood pressure. The continuous (i.e., smooth) nature of the background deflating cuff pressure affords the precise measurement of pulsatile waveforms, upon which are based the estimates of diastolic and systolic blood pressure.

The device 130 performs compression and decompression via a digitally-controlled DC motor and gearbox. To change speeds of compression and decompression, the device 130 employs pulse width modulation (PWM). PWM cyclically turns the motor on for some percentage of the cycle (i.e., the duty cycle) and off for the rest of the cycle. Thus, the compression and decompression background forces are not continuously changed, instead they are changed discretely via the PWM. This creates the discontinuous background pressure measurement illustrated in FIG. 4. From a signal processing perspective, it is difficult to separate the discrete stress changes caused by the motor-initiation and subsequent visco-elastic response effects, from the desired pulsatile arterial blood pressure data (again, see FIG. 4). Thus, the precision of the estimates of pulsatility required for determining systolic and diastolic pressure are hindered by these signal discontinuities. One could determine the exponential decay kinetics of the ear-vise assemblage, and subtract these from the measured waveform, in hope of obtaining a measure of the pulsatility free from visco-elastic discontinuities. However, every unique new device would have a different visco-elastic response kinetic, and the real-time signal processing using a model of multi-exponential decay kinetics would be computationally intensive (and would require higher sampling rates for more precision).

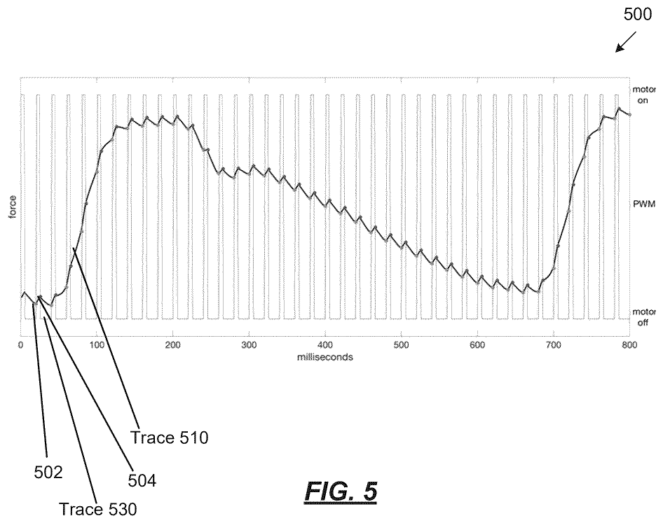

FIG. 5 is a chart 500 that shows an example sketch of visco-elastic moto-phase sampling (VEMPS) methodology for sampling arterial blood pressure in phase with pulse width modulation (PWM). VEMPS is a term used to describe a technique that affords the precise measurement of pulsatile waveforms from any device employing a solid polymeric (visco-elastic) clip, such as the visco-elastic clip. VEMPS removes the need for a pneumatic pressure pump (thus reducing size), and allows the pulsatile waveform estimates to proceed using any solid clip material/geometry and a computer-controlled DC motor/gearbox. Moreover, the technique requires no modelling of visco-elastic kinetics, minimizes real-time signal processing, is temperature independent, is solid material/geometry independent, and can work at all PWM motor speeds.

VEMPS works by phase-locking the sampling of the force sensor to a particular phase of the PWM signal. In an implementation, a device, such as the device 130, uses a 50 Hz PWM frequency to control the ear-vise motor speed; this corresponds to a 20 ms PWM cycle length (see e.g., FIG. 5). For VEMPS to work, the device must also use a 50 Hz analog to digital sampling frequency. This is convenient for the device because the onboard 24-bit sigma-delta analog to digital converter (ADC) has built-in digital low-pass filtering at 60 Hz and 50 Hz (designed around North American and UK home AC power grid frequencies, respectively). Moreover, the ADC can be controlled by an external clock, thus allowing for perfect synchronization of PWM motor-on events and ADC sampling. When the ADC sampling and ear-vise motor-on phases are synchronized, all the visco-elastic response effects are cancelled, (because the visco-elastic response kinetics are constant, see FIG. 3). Thus, the ADC is repeatedly sampling at the same phase of PWM for every motor-on event (see FIG. 5). Thus, the current device uses VEMPS to negate the visco-elastic response discontinuities.

VEMPS allows for a sampling of a very precise stress/pressure waveform which can be used to accurately estimate real-time pulsatility, and thus real-time oscillometric diastolic and systolic blood pressures. In doing so, VEMPS allows the design to use any clip configuration or material, provided the material visco-elastic properties stay constant over the given stress/strain range. Essentially, VEMPS allows the design to use a solid clip as the force providing medium, and a digitally controlled gearbox motor for precise control. Moreover, VEMPS works during compression and decompression, allowing for two precise measurements of arterial pulsatility waveforms per calibration run. Additionally, VEMPS does not require the high sample-rate that would be needed for real-time accurate modelling of visco-elastic response behavior; this greatly reduces the number of floating point computations and thus greatly reduces battery consumption.

Thus, FIG. 5 shows a chart of VEMPS methodology for sampling arterial blood pressure in phase with PWM. The trace 510 is postulated force across the ear-vise showing visco-elastic response to PWM of the ear-vise motor (the trace 530). The light circles 502 show the start of ear-vice motor-on; the dark circles 504 show the end of motor-on and the time when ADC sample taken. By sampling the force curve at the end of the motor-on, the visco-elastic decay kinetics during motor-off do not contribute to the variability of the measured sample.

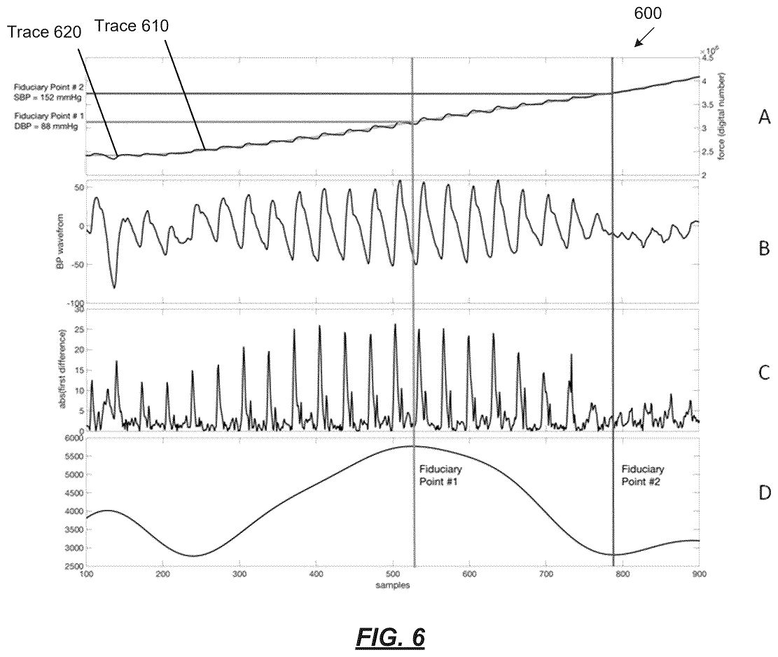

With respect to determining maximum pulsatility pressure and occlusion pressure, FIG. 6 is a chart 600 of an example signal processing methodology that could be used to precisely locate the fiduciary points.

The determination of diastolic blood pressure (DBP) and systolic blood pressure (SBP) requires two fiduciary points estimated from the force data acquired during steadily increasing compression of the ear tissue via the ear-vise. FIG. 6 shows the typical force data obtained during steadily increasing ear-vise compression. As applied compression force steadily increases, the pulsatility of the arterial blood pressure waveform (or a proxy therefor) reaches a point of maximum pulsatility (fiduciary point 1). At this point, the arterial blood pressure waveform (or a proxy therefor) has its maximum amplitude. As applied compression force increases further, the pulsatility of the arterial blood pressure waveform (or a proxy therefor) decreases until it reaches some minimum magnitude (fiduciary point 2). This point is referred to as the point of occlusion. There are several signal processing methodologies that could be used to precisely locate the fiduciary points. FIG. 6 is an illustration of one such methodology.

Real-time signal processing is used to locate the fiduciary points during a blood pressure determination compression run. FIG. 6 shows that the absolute value of the first-difference of the acquired force data (i.e., current sample force minus previous sample force) can be used, with further signal processing, to determine the two fiduciary points. FIG. 6 shows that a low-pass digital filter applied to the absolute of the first-difference data reveals the two fiduciary points: 1) the local maximum (fiduciary point 1), followed by 2) the next local minimum (fiduciary point 2). These two fiduciary points can then be used to estimate the ear-vise pressure of maximum pulsatility and occlusion. FIG. 6 shows the two fiduciary points projected onto the background force curve to obtain the measured force (and stress, i.e., measured force/ear-vise area). The background force curve is obtained via digital low-pass filtering of the force data sequence acquired during the compression run.

The measured force/stress at the two fiduciary points represent the device estimate of DBP and SBP for the individual subject during the time of measurement. For a large sample population, these device estimates are regressed against corresponding validated arm-cuff blood measurements to yield regression coefficients that can be used to determine standardized DBP and SBP measurements.

More particularly, in panel A of FIG. 6, the trace 610 is raw force data acquired during compression run, and the trace 620 is background force curve. Panel B shows the BP pulsatility curve during compression, showing maximum pulsatility and occlusion. Panel C shows the absolute value of the first differences of the raw force data. Panel D shows the results of low-pass digital filtering of the trace in panel C. The maximum is the fiduciary point #1 which is the point of maximum pulsatility. The minimum is the location of fiduciary point #2 which is the point of occlusion. These two points when projected on the background force curve yield the device estimate of diastolic and systolic blood pressure, respectively.

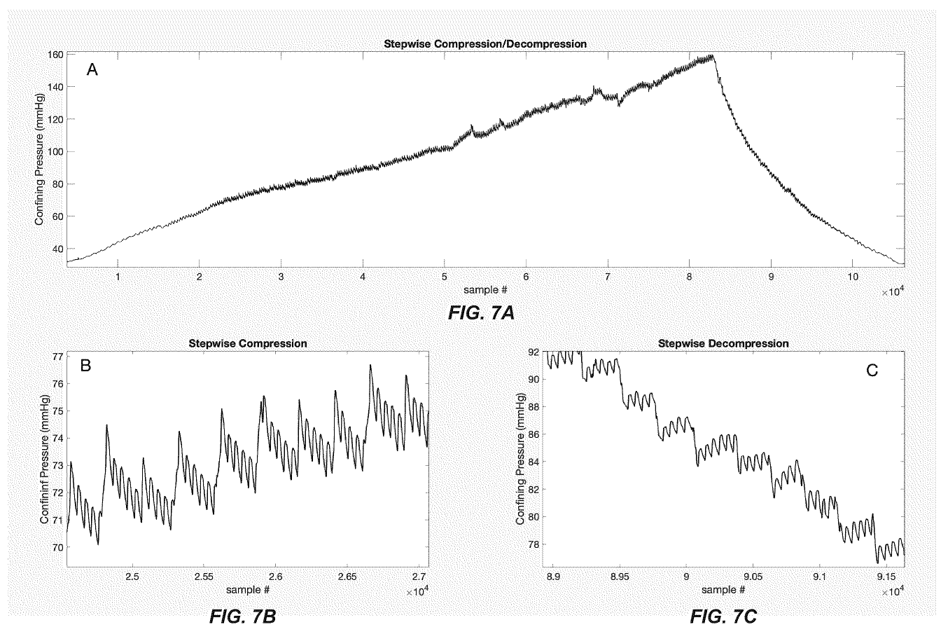

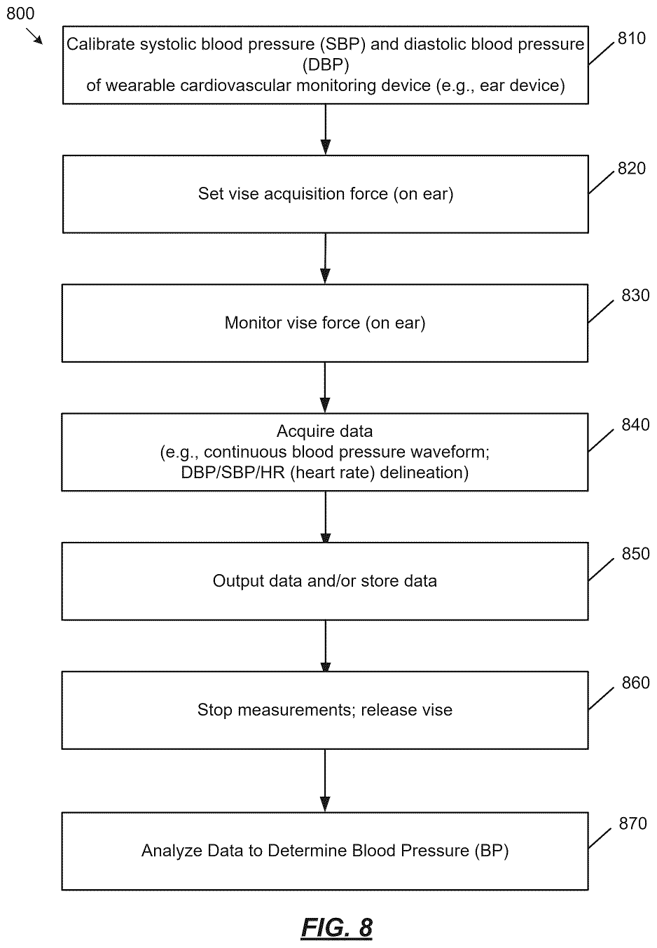

FIGS. 7A-7C illustrate another way to determine SBP and DBP by systematically performing a stepwise compression/decompression of ear tissue while measuring predetermined waveform parameters at each step. Predetermined waveform parameters, and the mmHg interval between the steps, can be determined using a Machine Learning (ML) algorithm applied to a learning set of stepwise compression/decompression sequences paired with a matching truth set of gold-standard SBP and DBP measurements. Stepwise compression/decompression is accomplished by systematically activating the Ear-vise 131 motor gearbox for varying durations in the forward direction to obtain a compression step (FIG. 7B), and in the reverse direction to obtain a decompression step (FIG. 7C). The duration and step start times can be determined empirically, again using a population of test subjects to measure and determine the best-fit (on a population basis) firmware function which provides step duration times for given average confining pressure values.

The waveform parameters, as determined by the ML algorithm, are eventually used in the real-time firmware implementation of the stepwise compression/decompression determination of each users DBP and SBP. In doing so, the ML parameters are evaluated at each step, and the ensemble step data are mathematically combined to determine the real-time DBP and SBP.

In some implementations, tissue compression does not occur at the ear, a compression drive mechanism is not at the ear, and a sensor is at the ear. In an implementation, the only thing mounted at the ear is the sensor/analog sampler/Bluetooth radio/micro-controller/flash RAM. Such an implementation has no mechanical workings mounted on the ear. Instead, the ear-mounted device is a passive ear clip designed to maintain a comfortable confining pressure at less than diastolic pressure (e.g., 30 mmHg). The clip contains the sensors and samples the sensor data for storage and/or radio/wire transmission.

This implementation works via a process of intermittent remote calibration. This process intermittently uses a compression drive and tissue/arterial compression at another body location, in conjunction with the continuous data obtained by the ear device. At intermittent times, the user might use ear device 130, a finger cuff BP device, or an upper arm inflationary cuff BP device to determine the “standard” BP. At this same time, the BP device transmits these latest BP measurements to the ear device (via radio or wire) such that the continuous pulsatility data obtained at the ear can be calibrated to match the “standard” BP data obtained at the other body location. Thus, the continuous ear data is intermittently and remotely calibrated. Moreover, if the inflatable finger cuff device is on the same side of the arterial tree as the ear clip, then intermittently also obtain simultaneous recordings at both sites; moreover, the ear and the fingertip are from the same branch of the arterial tree. The ear is more central representation of BP whereas the fingertip is much more distal representation of BP. The distal fingertip BP waveform is also more prone to a myriad of confounding augmentation waveforms from pressure reflections along the long arterial pathway. Additionally, the simultaneous recordings from the central and distal portions of the artery provide an opportunity to perform a transfer function analysis of the impedance properties of the arterial pathway. Moreover, one could also assess the travel times of the pulsatile waveform from the ear to fingertip, and this could provide other hitherto unknown pulse velocity data, which can be used for advanced assessment of cardiovascular wellness. It is noted that implementations described herein produce waveforms more akin to the aortic waveform. The central waveforms are the truest waveforms, without distortion from rebound waves from peripheral vessels in places like the fingers. Advantages include, for example: increased accuracy at predicting clinical outcomes such as stroke, heart attack, and death; no need for modelling to get central arterial pressure; and it is more straightforward to calculate stroke volume and peripheral vascular resistance from this pure signal, which in turn permits precision medicine treatment with different drug classes for heart failure and hypertension, and from hundreds of miles away.

In an embodiment of intermittent remote calibration, a smart phone protective case has a molded latex finger port, or a flip-up finger port, and upon fingertip insertion, an algorithm controls a small gearbox motor that adjusts a confining pressure applied between the nail bed and the finger tissue to determine systolic and diastolic BP, all the while in radio connection with the passive ear cuff and the smart phone. At this time, the ear cuff data gets remotely calibrated, and the transfer function between the ear and fingertip is determined and stored for future analysis. Via radio link (e.g., BluetoothLE), the ear clip and the smart phone can then inform the user when another remote calibration is necessary, or the user can perform the remote calibration at their discretion.

Another embodiment uses a device similar in size to a USB memory stick. The gearbox motor is mounted inside the device, and the conventional USB interface becomes a high speed serial interface for data download, and a charging port for the device and for the ear clip. This device would not require a smart phone.

With respect to the intermittent remote calibration devices described above, by removing the motor/gearbox and ear-vise from the ear site itself, the entire device at the ear becomes an order of magnitude smaller in size and mass. This affords a much more stable platform. Moreover, the power consumption at the ear is also greatly reduced allowing for longer wearing times between battery recharges. Furthermore, by intermittently having sensors at both ends of the arterial vascular tree (i.e., the ear and the fingertip), this allows for real-time intermittent determination of measured vascular impedance (as opposed to theoretical impedance offered by others), something unique to the field. In addition, this multi-sensor platform (albeit intermittent in the described embodiment) is a first step into the multi-sensor domain. The appeal of the ultra-small and ultra-light ear sensor, used intermittently in conjunction with a tiny memory-stick sized USB device is not to be overlooked. This industry-wide move into multi-sensor wearables is going to eventually happen; the intermittent remote calibration arrangement described above is one feasible step in this direction.

FIG. 8 is an operational flow of another implementation of a method 800 for blood pressure measurement with a wearable cardiovascular monitoring device, such as the ear device 130, for example.

At 810, the device (e.g., the ear device 130) is calibrated. For example, the SBP and the DBP is calibrated by firmware of the device, pursuant to a start command being received (e.g., the start 111 input of an external software input 110).

At 820, the vise acquisition force (e.g., for the ear) is set. This may be set to a predetermined amount or set to an amount pursuant to a predetermined technique or algorithm.

At 830, force is applied (e.g., to the ear) and monitored to ensure that the force is maintained at the proper amount from 820.

At 840, data is acquired by the device. Depending on the implementation, data may include a continuous blood pressure waveform (or a proxy therefor), and/or DBP, SBP, and HR delineation.

At 850, the data that was acquired at 840 may be stored (e.g., in memory or storage of the device, such as flash RAM or other local or external storage). Alternatively, or additionally, the data may be outputted or displayed to one or more other devices, for example.

At 860, the measurements stop (e.g., pursuant to a stop 112 input being received), and the vise is released.

At 870, the stored and/or outputted data may be processed using, for example, a machine learning-based systolic blood pressure predicting model using data collected by the ear device 130 may be used. A first Support Vector Machine (SVM) regression model may be built to determine the systolic blood pressure. In addition to the average between the measured ear lobe temperature before and after data recording, some features are extracted from the BP waveforms recorded by the ear device 132. A non-limiting listing of the features may be the following:
The average of the pressure at one of the three points where the mean of the inflation and deflation and/or compression and decompression curves change most significantly, respectively.
The confining pressure when there is a rapid change in the trend of signal oscillations during decompression.
E The pressure when a high increase in signal oscillations amplitude during decompression is observed.
The maximum oscillation amplitude during deflation.
The coefficient representing the fitted polynomial of degree 1 to the decompression curve.
The median frequency of the deflation data.
The mean of the difference of successive points during deflation.

A machine learning-based diastolic blood pressure predicting model may also be used. A second algorithm may be used that is a Support Vector Machine (SVM) regression model intended to estimate diastolic blood pressure. Similar to the first ML-Based systolic blood pressure predicting model above, the characteristics used in this model may include, but are not limited to, the mean ear lobe temperature, alongside. Other non-limiting descriptors extracted from the recorded compression and decompression runs may include the following:
The average of the pressure at one of the three points where the mean of the and/or compression and decompression curves change most significantly, respectively.
The confining pressure when there is a rapid change in the trend of signal oscillations during decompression.
The maximum oscillation amplitude during deflation.
The median frequency of the deflation data.
The mean of the difference of successive points during deflation.
The mean of the decompression curve.
The peaks median of the decompression curve.
The mean of pressures where a change is detected in mean and slope during the decompression curve.

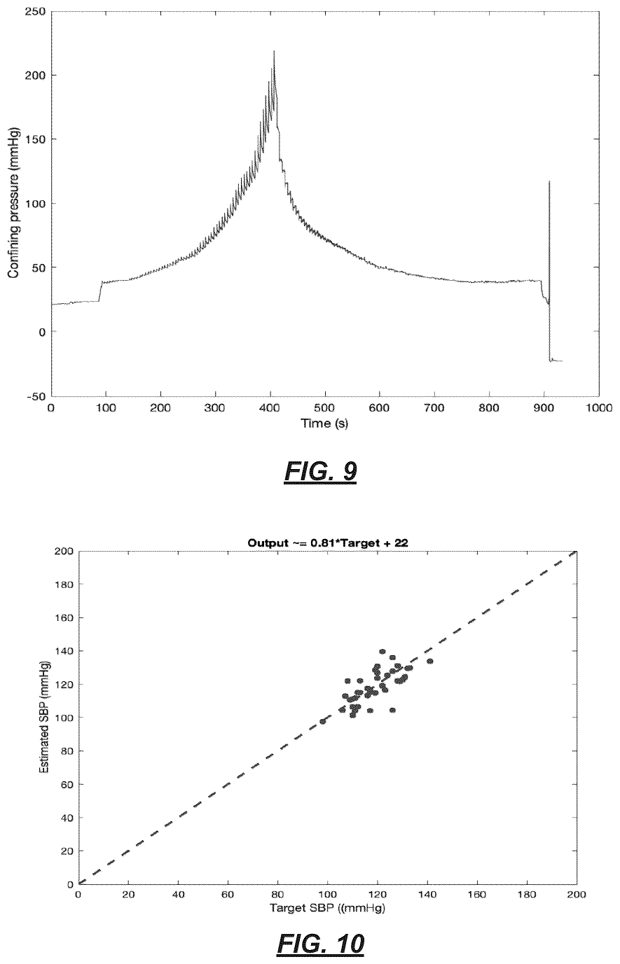

With reference to FIGS. 9-13, experimental results of the machine-learning based model that may be used to generate BP data collected by the device 130 are shown. In a study, 81 subjects were enrolled for data collection using our new ear device (see, FIG. 9). Some vital signs were first recorded like age, sex, weight and height. The ear lobe temperature and two arm cuff readings are taken before and after recording of compression and decompression runs. The mean of these systolic and diastolic arm cuff pressures will be the target SBP and DBP. 41 recorded BP waveforms will be used as training set to train the machine learning models for SBP and DBP estimation and the left 40 signals will be used as test set to assess the performance of the two estimation algorithms.

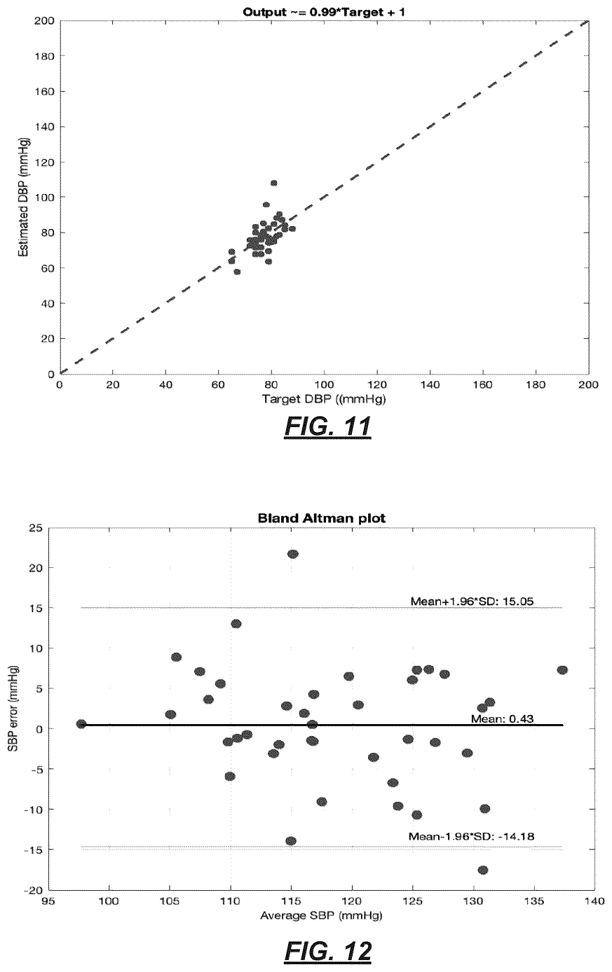

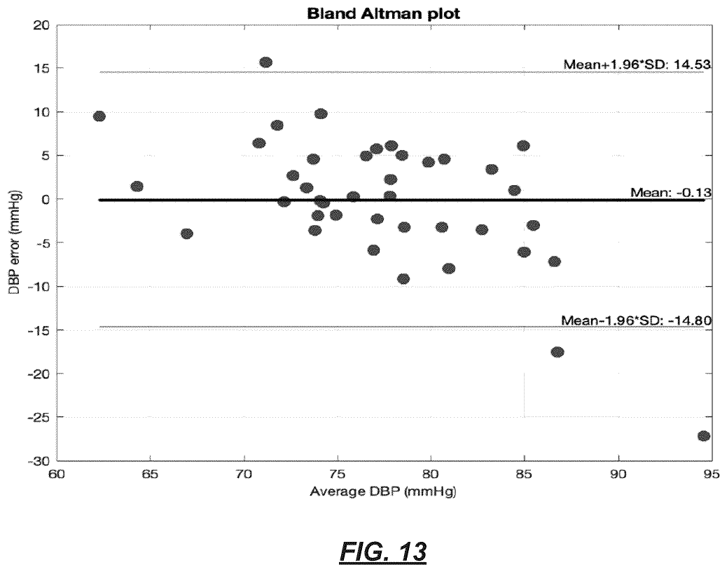

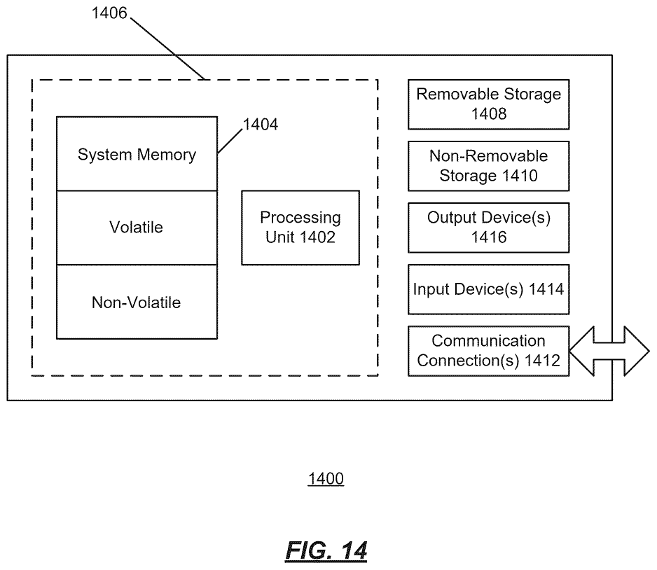

FIGS. 10-11 illustrate the scatter plot of arm cuff SBP vs. estimated SBP and arm cuff DBP vs. estimated DBP for the test set, respectively. To compare the estimation results of our device with the conventional arm cuff readings, FIGS. 12-13 show bland Altman plots respectively for systolic and diastolic blood pressure. As illustrated in the Table 1, below, the ear device measurements produce a mean error of 0.43 mmHg and a standard deviation error of 7.46 for systolic blood pressure and a mean error of −0.13 mmHg and a standard deviation error of 7.48 for diastolic blood pressure.

TABLE 1

| Pressure Measurement | Mean error | Standard deviation error |
|---|---|---|
| SBP | 0.43 | 7.46 |
| DBP | −0.13 | 7.48 |

FIG. 14 shows an exemplary computing environment in which example embodiments and aspects may be implemented. The computing device environment is only one example of a suitable computing environment and is not intended to suggest any limitation as to the scope of use or functionality.

With reference to FIG. 14, an exemplary system for implementing aspects described herein includes a computing device, such as computing device 1400. In its most basic configuration, computing device 1400 typically includes at least one processing unit 1402 and memory 1404. Depending on the exact configuration and type of computing device, memory 1404 may be volatile (such as random access memory (RAM)), non-volatile (such as read-only memory (ROM), flash memory, etc.), or some combination of the two. This most basic configuration is illustrated in FIG. 14 by dashed line 1406.

Computing device 1400 may have additional features/functionality. For example, computing device 1400 may include additional storage (removable and/or non-removable) including, but not limited to, magnetic or optical disks or tape. Such additional storage is illustrated in FIG. 14 by removable storage 1408 and non-removable storage 1410.

Computing device 1400 typically includes a variety of computer readable media. Computer readable media can be any available media that can be accessed by the device 1400 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 1404, removable storage 1408, and non-removable storage 1410 are all examples of computer storage media. Computer storage media include, but are not limited to, Read-Only Memory (ROM) and/or Random-Access Memory (RAM), electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 1400. Any such computer storage media may be part of computing device 1400.

Computing device 1400 may contain communication connection(s) 1412 that allow the device to communicate with other devices. Computing device 1400 may also have input device(s) 1414 such as a keyboard, mouse, pen, voice input device, touch input device, etc. Output device(s) 1416 such as a display, speakers, printer, etc. may also be included. All these devices are well known in the art and need not be discussed at length here.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A method for determining blood pressure, comprising:
receiving, by one or more processors, blood pressure data acquired from a wearable cardiovascular monitoring device configured to be worn on an ear of a subject;
extracting, from the blood pressure data, a plurality of waveform-based features;
determining a systolic blood pressure value of the subject by inputting at least a subset of the waveform-based features into a machine learning-based systolic blood pressure model; and
determining a diastolic blood pressure value of the subject by inputting at least a subset of the waveform-based features into a machine learning-based diastolic blood pressure model.

2, The method of claim 1, wherein extracting the plurality of waveform-based features comprises extracting one or more features from at least one of an inflation curve, a deflation curve, a compression curve, a decompression curve, a compression run, and a decompression run of the blood pressure data.

3. The method of claim 1, wherein extracting the plurality of waveform-based features comprises:
identifying, from the blood pressure data, at least one point at which a mean of an inflation curve and a deflation curve or a compression curve and a decompression curve changes most significantly; and
computing an average pressure at the at least one point.

4. The method of claim 1, wherein extracting the plurality of waveform-based features comprises computing a confining pressure at which there is a rapid change in a trend of signal oscillations during decompression.

5. The method of claim 1, wherein extracting the plurality of waveform-based features comprises computing at least one of a pressure at which a high increase in signal oscillation amplitude during decompression is observed; a maximum oscillation amplitude during deflation; and a median frequency of deflation data.

6, The method of claim 1, wherein extracting the plurality of waveform-based features comprises computing a coefficient of a fitted polynomial of degree 1 to a decompression curve of the blood pressure data.

7. The method of claim 1, wherein extracting the plurality of waveform-based features comprises computing a mean of differences of successive points in the blood pressure data during deflation.

8. The method of claim 1, wherein determining the systolic blood pressure value comprises inputting into the machine learning-based systolic blood pressure model features comprising:
a confining pressure at which there is a rapid change in a trend of signal oscillations during decompression;
a pressure at which a high increase in signal oscillation amplitude during decompression is observed;
a maximum oscillation amplitude during deflation;
a coefficient of a fitted polynomial of degree 1 to a decompression curve;
a median frequency of deflation data; and
a mean of a difference of successive points during deflation.

9. The method of claim 1, wherein determining the diastolic blood pressure value comprises inputting into the machine learning-based diastolic blood pressure model features comprising:
an average pressure at one of three points where a mean of inflation and deflation or compression and decompression curves changes most significantly;
a confining pressure at which there is a rapid change in a trend of signal oscillations during decompression;
a maximum oscillation amplitude during deflation;
a median frequency of deflation data;
a mean of a difference of successive points during deflation;
a mean of a decompression curve;
a peaks median of the decompression curve; and
a mean of pressures where a change is detected in mean and slope during the decompression curve.

10. The method of claim 1, further comprising determining an average ear lobe temperature from temperature measurements obtained before and after data recording, and providing the average ear lobe temperature as an input feature to at least one of the machine learning-based systolic blood pressure model and the machine learning-based diastolic blood pressure model.

11. The method of claim 1, wherein the machine learning-based systolic blood pressure model and the machine learning-based diastolic blood pressure model each comprise a Support Vector Machine regression model trained using a plurality of training examples of waveform-based features paired with corresponding arm cuff systolic and diastolic blood pressure measurements.

12. The method of claim 11, further comprising:
dividing a set of recorded blood pressure data into a training subset and a test subset;
training the Support Vector Machine regression models using the training subset; and
validating performance of the Support Vector Machine regression models using the test subset.

13. The method of claim 1, wherein determining the systolic blood pressure value and determining the diastolic blood pressure value are performed in real time on waveform data acquired during a stepwise compression and decompression sequence implemented by the wearable cardiovascular monitoring device.

14. The method of claim 13, wherein the stepwise compression and decompression sequence is generated by a motor-driven ear-vise that applies discrete changes in confining pressure to ear tissue, and wherein the discrete changes and timing of compression and decompression steps are selected based on parameters optimized using the machine learning-based models.

15. A system for determining blood pressure, comprising:
a wearable cardiovascular monitoring device configured to be worn on an ear of a subject and to generate blood pressure data; and
one or more processors coupled to a memory storing instructions which, when executed by the one or more processors, cause the system to:
receive the blood pressure data from the wearable cardiovascular monitoring device;
extract a plurality of waveform-based features from the blood pressure data;
determine a systolic blood pressure value using a machine learning-based systolic blood pressure model applied to at least a subset of the waveform-based features; and
determine a diastolic blood pressure value using a machine learning-based diastolic blood pressure model applied to at least a subset of the waveform-based features.

16, The system of claim 15, wherein the wearable cardiovascular monitoring device comprises an ear-vise configured to apply a confining pressure to ear tissue and at least one force sensor configured to measure oscillatory changes associated with arterial blood pressure.

17. The system of claim 15, wherein the one or more processors are further configured to:
control the wearable cardiovascular monitoring device to execute a stepwise compression and decompression sequence; and
time extraction of the waveform-based features to discrete compression and decompression steps of the sequence.

18. A non-transitory computer-readable medium storing instructions which, when executed by one or more processors, cause the one or more processors to perform operations comprising:
receiving blood pressure data acquired from a wearable cardiovascular monitoring device configured to be worn on an ear of a subject;
extracting a plurality of waveform-based features from the blood pressure data;
providing at least a first subset of the waveform-based features as input to a trained machine learning-based systolic blood pressure model to generate an estimated systolic blood pressure value; and
providing at least a second subset of the waveform-based features as input to a trained machine learning-based diastolic blood pressure model to generate an estimated diastolic blood pressure value.

19. The non-transitory computer-readable medium of claim 18, wherein the operations further comprise receiving arm cuff blood pressure measurements and, using the arm cuff blood pressure measurements together with the waveform-based features, updating at least one of the machine learning-based systolic blood pressure model and the machine learning-based diastolic blood pressure model.

20. The non-transitory computer-readable medium of claim 18, wherein the operations further comprise transmitting the estimated systolic blood pressure value and the estimated diastolic blood pressure value to an external device for storage, display, or further clinical analysis.
